(19) United States
(12) Patent Application Publication
(10) Pub. No.: US 2021/0378625 A1
(43) Pub. Date: Dec. 9, 2021

(54) STETHOSCOPE HEAD AND STETHOSCOPE ASSEMBLY HAVING THE SAME

(71) Applicant: VITALCHAINS CORPORATION, Menlo Park, CA (US)

(72) Inventors: TZU-CHIH LIN, Chupei, Hsinchu (TW); KUN-HSI TSAI, Zhubei, Hsinchu (TW)

(21) Appl. No.: 17/288,001

(22) PCT Filed: Oct. 23, 2018
(86) PCT No.: PCT/US18/56959

§ 371 Date: Apr. 23, 2021

(51) Int. Cl. A61B 7/04

(52) U.S. Cl. CPC A61B 7/04; A61B 2560/02; A61B 2560/04

(57) ABSTRACT

The present invention provides a stethoscope head switchable between a digital mode and a conventional mode. The stethoscope head comprises a housing, a chestpiece, a switching mechanism, a microphone, a speaker, and a first power switch. The microphone is configured for receiving acoustic signals from the chestpiece. The speaker is configured for playing the acoustic signals received by the microphone. The first power switch is configured to be engaged by the switching mechanism. The switching mechanism is configured for switching the stethoscope head between the digital mode and the conventional mode when engaged by the first power switch.

### FIELD OF THE INVENTION

The present disclosure relates to a stethoscope, and more particularly, to a stethoscope head switchable between a digital mode and a conventional mode.

### BACKGROUND OF THE INVENTION

Auscultation has long been a very useful tool for medical diagnosis of ailments. By using a stethoscope, healthcare professionals can listen to and identify sounds associated with abnormalities. Conventional stethoscopes operate solely on the principles of sound travel and the coupling of sound waves through an acoustic cavity. Electronic or digital stethoscopes, on the other hand, incorporate digital and electronic functions to stethoscopes, therefore improving sound output in auscultation. However, existing digital stethoscopes are susceptible to failure in the event of electronic or battery failure, and operation of a digital stethoscope is also limited by its battery capacity. For example, in cases where battery drain or electronic failure occurs during digital auscultation, significant inconvenience would result if the healthcare professional operating the digital stethoscope does not have another stethoscope on hand. Accordingly, while becoming popular nowadays, digital stethoscopes still cannot replace conventional stethoscopes completely.

### BRIEF SUMMARY OF THE INVENTION

In view of the above-described shortcomings, an objective of the present disclosure is to provide a stethoscope adapted to operate in both digital and conventional modes.

The present invention provides a stethoscope head switchable between a digital mode and a conventional mode. The stethoscope head comprises a housing, a chestpiece, a switching mechanism, a microphone, a speaker, and a first power switch. The chestpiece is disposed below the housing and comprising a through hole at a center of the chestpiece. The switching mechanism is disposed in the housing and above the chestpiece, and configured for switching the stethoscope head between the digital mode and the conventional mode. The microphone is disposed in the housing and configured for receiving acoustic signals from the through hole of the chestpiece. The speaker is disposed in the housing and configured for playing the acoustic signals received by the microphone. The first power switch is disposed in the housing and configured to be engaged by the switching mechanism.

Overall, the present invention provides the stethoscope head adapted to operate in both digital and conventional modes, such that overcomes the inconvenience of digital stethoscope alone with battery drain or electronic failure.

### BRIEF DESCRIPTION OF THE DRAWINGS

The accompanying drawings illustrate one or more embodiments of the present invention and, together with the written description, explain the principles of the present invention. Wherever possible, the same reference numbers are used throughout the drawings referring to the same or like elements of an embodiment.

FIG. 1 is a perspective view of a stethoscope assembly in a digital mode in accordance with an embodiment of the present disclosure;

FIG. 2 is a perspective view of a stethoscope assembly in a conventional mode in accordance with the embodiment of the present disclosure;

FIG. 3 is an exploded view of a stethoscope head of the stethoscope assembly in accordance with the embodiment of the present disclosure;

FIG. 4 is a perspective view of a switching mechanism of the stethoscope head in accordance with the embodiment of the present disclosure;

FIG. 5 is a cross-sectional view of the stethoscope head in the digital mode in accordance with the embodiment of the present disclosure;

FIG. 6 is another cross-sectional view of the stethoscope head in the digital mode in accordance with the embodiment of the present disclosure;

FIG. 7 is another cross-sectional view of the stethoscope head in the conventional mode in accordance with the embodiment of the present disclosure;

FIG. 8 is a perspective view of a stethoscope head in a digital mode in accordance with another embodiment of the present disclosure;

FIG. 9 is a perspective view of the stethoscope assembly in a conventional mode in accordance with the other embodiment of the present disclosure;

FIG. 10 is an exploded view of the stethoscope head in accordance with the other embodiment of the present disclosure;

FIG. 11 is a perspective view of a housing with a microphone installed in accordance with the other embodiment of the present disclosure;

FIG. 12 is a cross-sectional view the stethoscope head in the digital mode in accordance with the other embodiment of the present disclosure;

FIG. 13 is a cross-sectional view of the stethoscope head in the conventional mode in accordance with the other embodiment of the present disclosure;

FIG. 14 is a perspective view of a switching mechanism of the stethoscope head in accordance with the other embodiment of the present disclosure;

FIG. 15 is a bottom view of the housing with the microphone and the switching mechanism in accordance with the other embodiment of the present disclosure;

FIG. 16 is a bottom view of the switching mechanism in accordance with the other embodiment of the present disclosure;

FIG. 17 is a rear view of the switching mechanism in accordance with the other embodiment of the present disclosure;

FIG. 18 is another perspective view of the switching mechanism in accordance with the other embodiment of the present disclosure;

FIG. 19 is a cross-sectional view of FIG. 9 in accordance with the other embodiment of the present disclosure;

FIG. 20 is another cross-sectional view of FIG. 9 in accordance with the other embodiment of the present disclosure;

FIG. 21 is a perspective view of a stethoscope assembly with a power switch in accordance with the other embodiment of the present disclosure;

FIG. 22 is a perspective view of the stethoscope assembly with the power switch in accordance with the other embodiment of the present disclosure;

FIG. 23 is a perspective view of a stethoscope assembly with two power switches in accordance with the other embodiment of the present disclosure; and

FIG. 24 is a perspective view of a stethoscope assembly with a phone jack module in accordance with the other embodiment of the present disclosure.

In accordance with common practice, the various described features are not drawn to scale and are drawn to emphasize features relevant to the present disclosure. Like reference characters denote like elements throughout the figures and text.

### DETAILED DESCRIPTION OF THE PREFERRED EMBODIMENTS

The present invention will now be described more fully hereinafter with reference to the accompanying drawings illustrating various exemplary embodiments of the invention. The present invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the terms “and/or” and “at least one” include any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

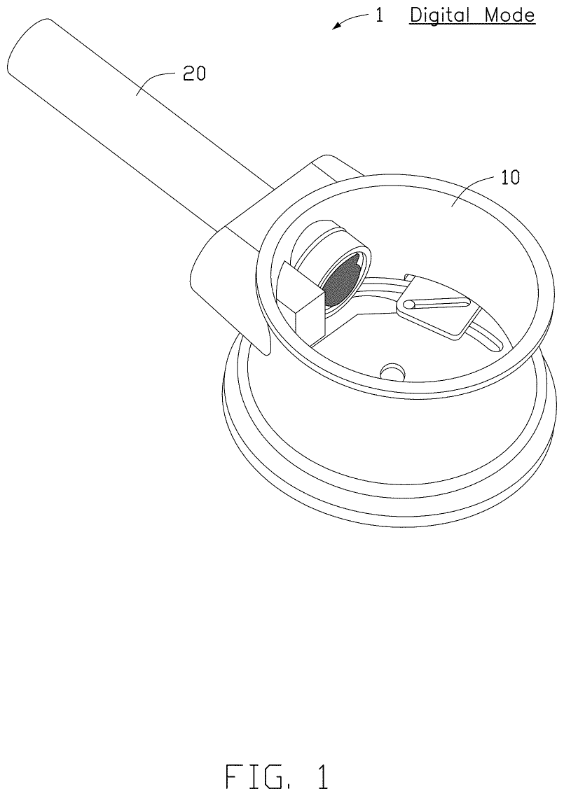

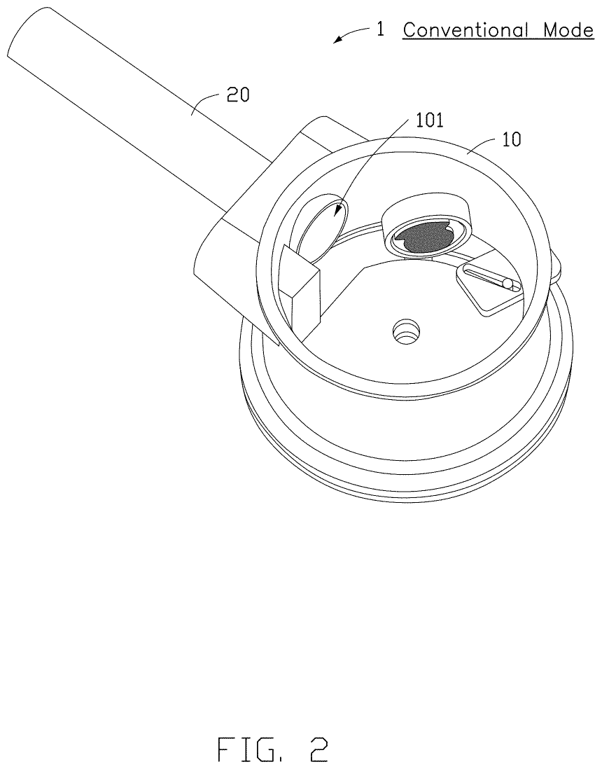

Referring to FIG. 1 and FIG. 2. A stethoscope assembly 1 according to one embodiment of the present disclosure includes a stethoscope head 10, a tube member 20, and at least one ear tip (not shown). The tube member 20 has a proximal end and a distal end. The proximal end is coupled to the ear tip, and the distal end is in fluid communication to a first opening 101 of the stethoscope head 10.

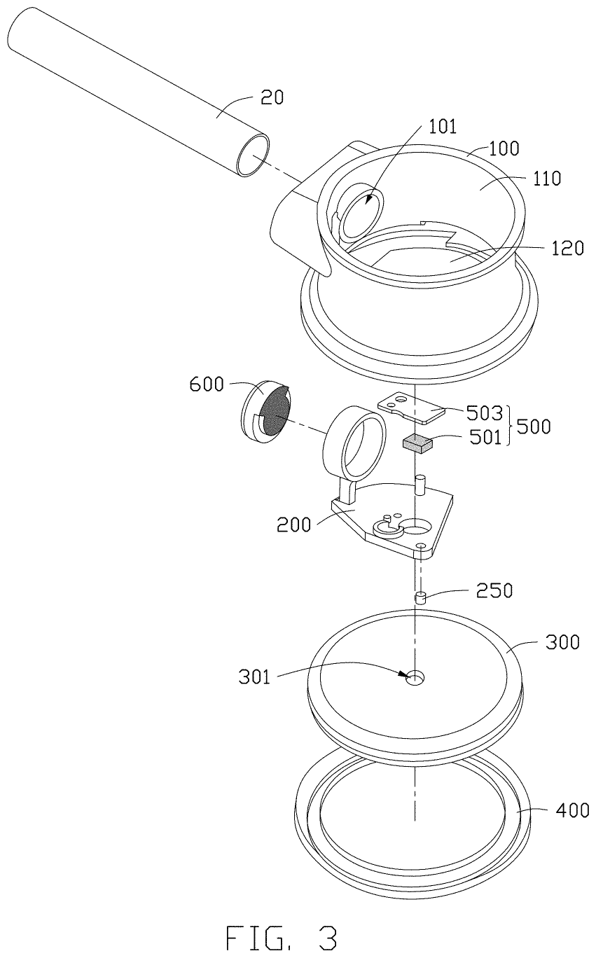

Referring to FIG. 3. In the embodiment, the stethoscope head 10 includes a cap (not shown), a housing 100, a switching mechanism 200, a chestpiece 300, a diaphragm 400, a microphone module 500, and a speaker 600. The housing 100 has a proximal end, a distal end, and a side wall 110 extended therebetween. The housing 100 further includes a first deck 120 and the first opening 101. The first deck 120 is substantially perpendicular to the side wall 110 and located between the proximal end and the distal end of the housing 100. The first opening 101 is located at the side wall 110 of the housing 100. The diaphragm 400 is coupled to the distal end of the housing 100. The switching mechanism 200 and the chestpiece 300 are disposed between the diaphragm 400 and the first deck 120, and the switching mechanism 200 is disposed between the first deck 120 and the chestpiece 300. The microphone module 500 includes a microphone 501 and a printed circuit board (PCB) 503. The microphone 501 is electrically connected to the PCB 503, and may be a micro-electronic mechanical system (MEMS) component. The speaker 600 is also electrically connected to the PCB 503, therefore allowing the acoustic signals to be received by the microphone 501 and sent to the speaker 600 via the PCB 503. It should be appreciated by person having ordinary skill in the art, that the acoustic signals enter the microphone 501 in a form of physiological sounds, for example: heartbeats, pulse sounds, wherein the acoustic signals are converted by the microphone 501 into digital signals, and therefore the acoustic signals can be played by the speaker 600.

In the embodiment, the stethoscope head 10 is switchable between a digital mode, as illustrated in FIG. 1, and a conventional mode, as illustrated in FIG. 2, by moving the switching mechanism 200 between a first state and a second state, correspondingly. As shown in FIGS. 1 and 3, the chestpiece 300 has a through hole 301 at the center. When the switching mechanism 200 is at the first state, the microphone 501 is exposed to the through hole 301, allowing the acoustic signals passing through the through hole 301 to be received by the microphone 501 and sent to the tube member 20 via the speaker 600, and hence causing the stethoscope head 10 to operate in the digital mode. Alternatively, as shown in FIG. 2, when the switching mechanism 200 is at the second state, the microphone 501 is blocked from the through hole 301, allowing the acoustic signals passing through the through hole 301 to travel to the tube member 20 directly without being received by the microphone module 500, and hence causing the stethoscope head 10 to operate in the conventional mode.

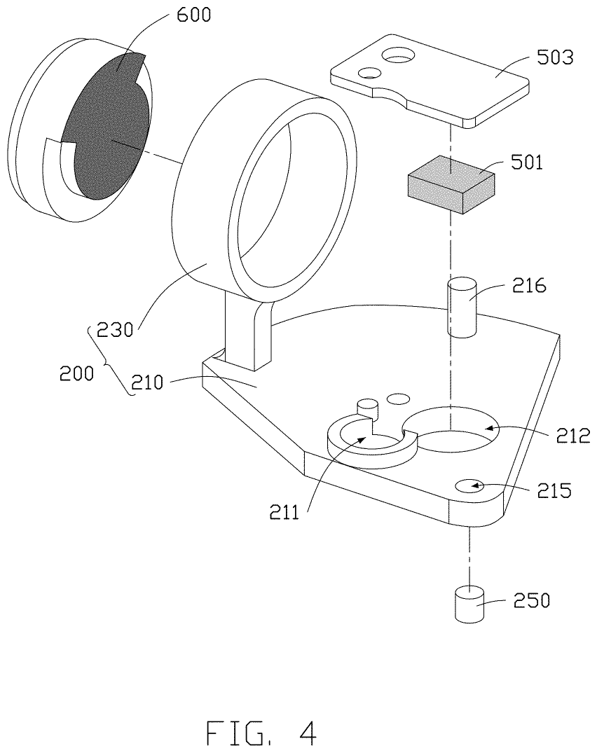

Referring to FIG. 4. In one embodiment of the present disclosure, the switching mechanism 200 is a substantially L-shaped component. The switching mechanism 200 include a first base 210 and a first extension 230 substantially perpendicular to the first base 210. The first base 210 includes a first hole 211 and a second hole 212; the second hole 212 is configured to accommodate the microphone 501. The first base 210 further includes a pin hole 215 and a first protrusion 216; the pin hole 215 is configured for accommodating a pin 250, to allow the first base 210 to rotate about the pin 250 between the first state and the second state.

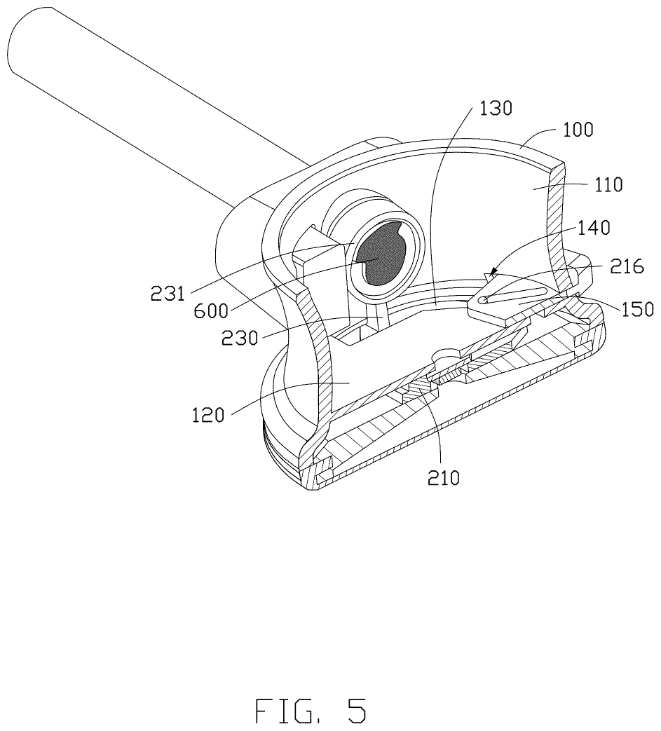

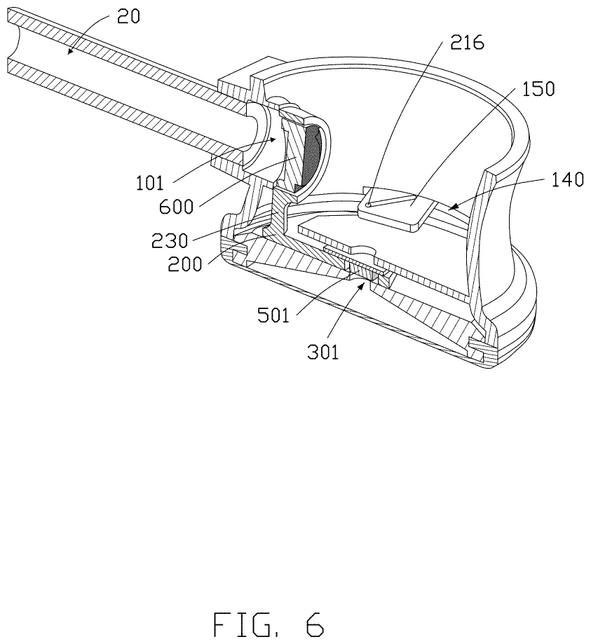

Referring to FIG. 4, FIG. 5 and FIG. 6. In the embodiment, the first deck 120 is only partially connected to the housing 100, thus forming a guiding structure 130 between the first deck 120 and the side wall 110 of the housing 100. In one embodiment, the guiding structure 130 may be a slit curved between a periphery of the first deck 120 and the side wall 110. The switching mechanism 200 and the housing 100 are so arranged that the first base 210 of the switching mechanism 200 is disposed below the first deck 120, and the first extension 230 and the first protrusion 216 of the switching mechanism 200 extend through the guiding structure 130, thus allowing the first extension 230 and the first protrusion 216 to move within the guiding structure 130 between the first state and the second state while the first base 210 rotates about the pin 250. The first extension 230 further includes a holder 231 configured to hold the speaker 600, therefore allowing the speaker 600 to move with the switching mechanism 200 along the guiding structure 130. In the embodiment, the housing 100 further includes a sliding structure 140 at the side wall 110 corresponding to the first protrusion 216, such that a lever 150 of the switching mechanism 200 connected to the first protrusion 216 can extend through and slide along the sliding structure 140; therefore, by sliding the lever 150 along the sliding structure 140, a user of the stethoscope assembly 1 can move the switching mechanism 200 between the first state and the second state. In one embodiment, the sliding structure 140 may be a slit curved along the side wall 110.

Referring to FIG. 6, together with FIG. 4. When the stethoscope head 10 is in the digital mode, the switching mechanism 200 is at the first state, the microphone 501 accommodated in the second hole 212 of the first base 210 of the switching mechanism 200 is exposed to the through hole 301 of the chestpiece 300, and the speaker 600 held by the first extension 230 is aligned with the first opening 101 of the housing 100. Therefore, the stethoscope head 10 receives the acoustic signals passing through the through hole 301 by the microphone 501 in the second hole 212, and the speaker 600 plays the acoustic signals into the tube member 20 through the first opening 101 of the housing 100.

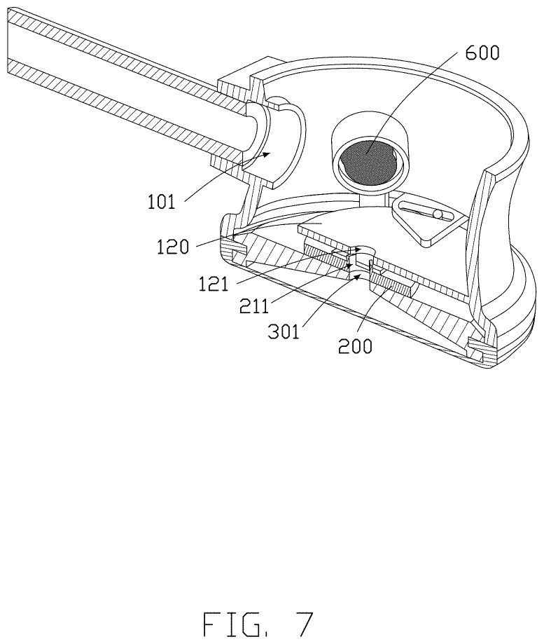

Referring to FIG. 7, together with FIG. 4. When the stethoscope head 10 is in the conventional mode, the switching mechanism 200 is at the second state, and the first hole 211 of the first base 210 of the switching mechanism 200 is aligned with the through hole 301 of the chestpiece 300 and a first aperture 121 disposed substantially at the center of the first deck 120. In such configuration, the microphone 501 is moved away and blocked from the through hole 301, and the speaker 600 on the first extension 230 is moved away from the first opening 101 of the housing 100. Therefore, the acoustic signals passing through the through hole 301 travels directly to the first opening 101 of the housing 100 via the first aperture 121 of the first deck 120 without reaching the microphone 501 nor the speaker 600.

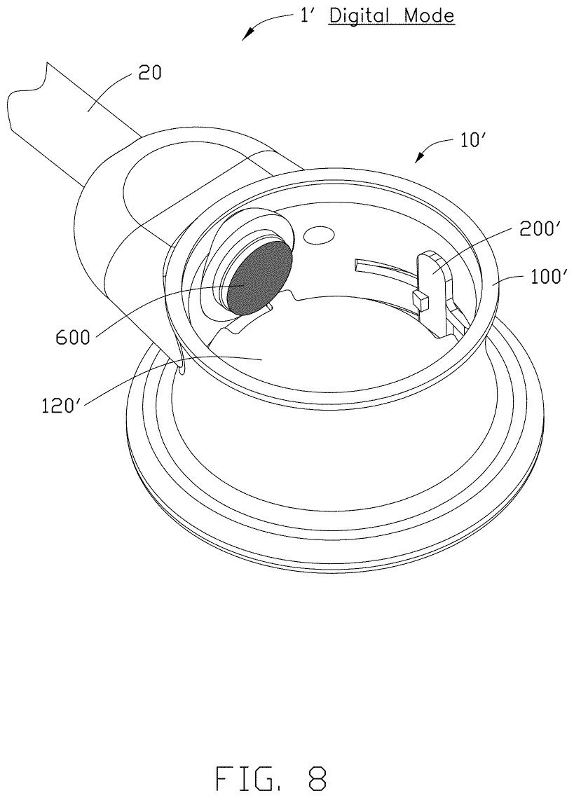

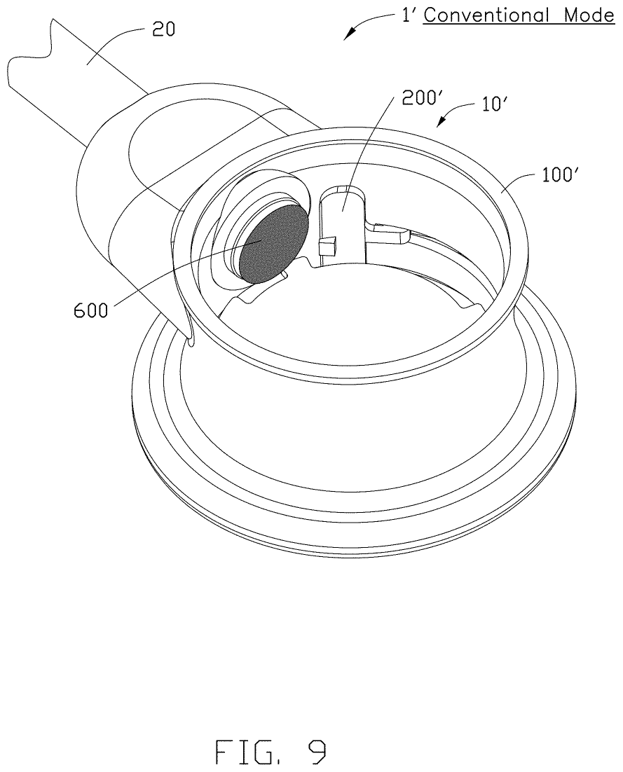

Referring to FIG. 8 to FIG. 10. According to another embodiment of the present disclosure, a stethoscope head 10′ is switchable between a digital mode, as illustrated in FIG. 8, and a conventional mode, as illustrated in FIG. 9. In the embodiment of the present disclosure, the stethoscope head 10′ includes the housing 100′, a switching mechanism 200′, the chestpiece 300, the diaphragm 400, a microphone module 500′, and the speaker 600. A user of a stethoscope assembly 1′ having the stethoscope head 10′ may switch modes of the stethoscope head 10′ by moving the switching mechanism 200′ between a first state and a second state.

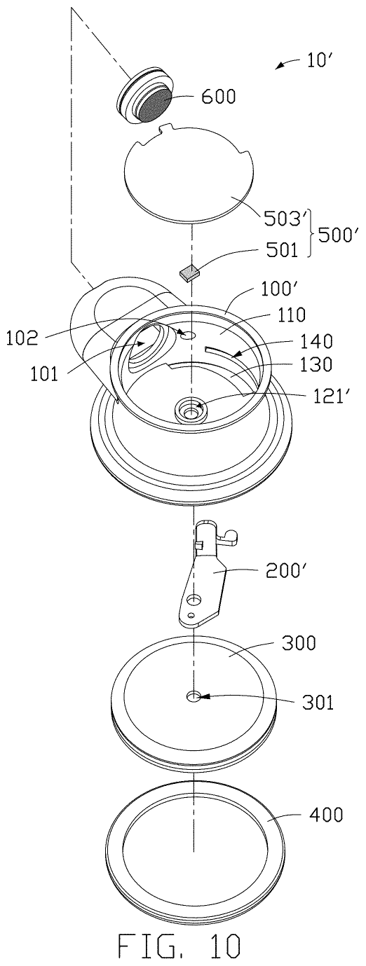

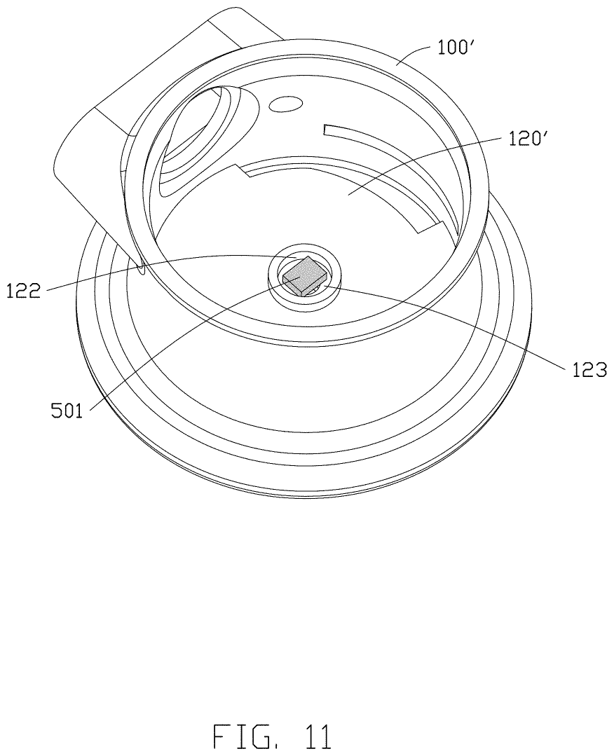

Referring to FIG. 10 together with FIG. 11. The microphone module 500′ includes the microphone 501 and a PCB 503′ electrically connected to the microphone 501. The microphone module 500′ is disposed above a second deck 120′ of the housing 100′. The second deck 120′ further includes an elevated structure 122 and a holding structure 123. The elevated structure 122 extends perpendicularly around a second aperture 121′ to stationarize the microphone 501 at the second aperture 121′ without horizontal displacement, for example, the elevated structure 122 may be a round-shaped protrusion on the second deck 120′; similarly, the holding structure 123 holds the microphone 501 within the second aperture 121′ to prevent the microphone 501 from falling, for example, the holding structure 123 may be a radial extension from the periphery of the second aperture 121′. In one embodiment, a diameter of the elevated structure 122 is equal to or larger than the second aperture 121′. The PCB 503′ is disposed within the housing 100 and above the microphone 501. The speaker 600 is disposed within the first opening 101 of the housing 100′ and is electrically connected to the PCB 503′ as well, hence allowing the acoustic signals received by the microphone 501 to be played by the speaker 600 via the PCB 503′.

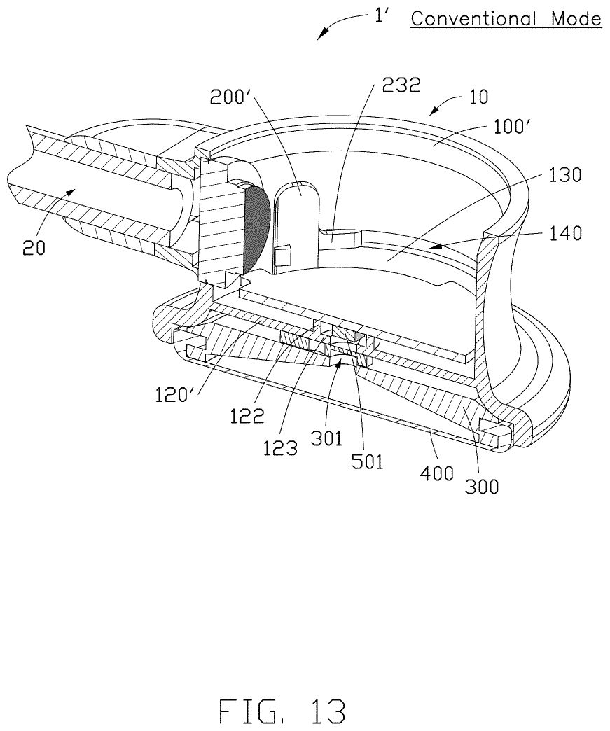

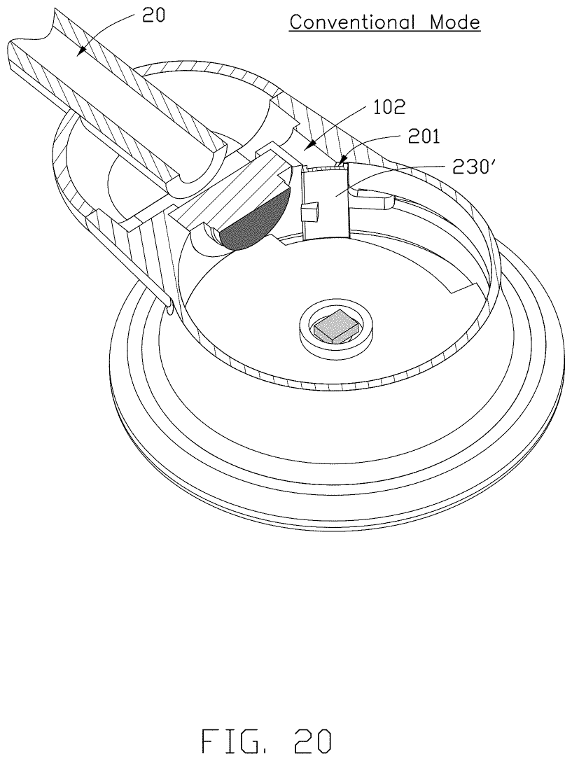

As illustrated in FIG. 10, the housing 100′ further includes a second opening 102 located at the side wall 110. It should be noted that, both the speaker 600 and the second opening 102 are in fluid communication with the tube member 20 (as illustrated in FIG. 20). The switching mechanism 200′, the chestpiece 300, and the diaphragm 400 are disposed below the second deck 120′ in the described order, such that the second aperture 121′ substantially aligns with the through hole 301 of the chestpiece 300. In the embodiment, the switching mechanism 200′ partially extends through the guiding structure 130 and the sliding structure 140 of the housing 100′ (as illustrated in FIG. 13). As aforementioned, the guiding structure 130 may be a slit curved between a periphery of the second deck 120′ and the side wall 110; the sliding structure 140 may be a slit curved along the side wall 110.

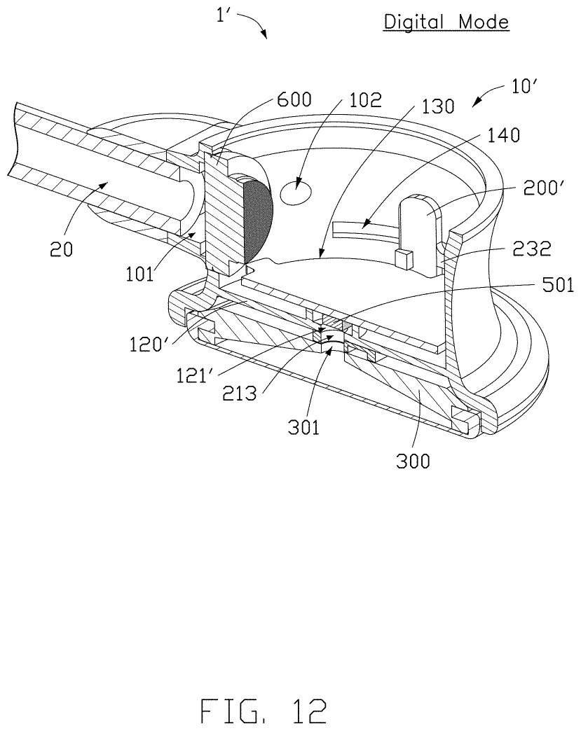

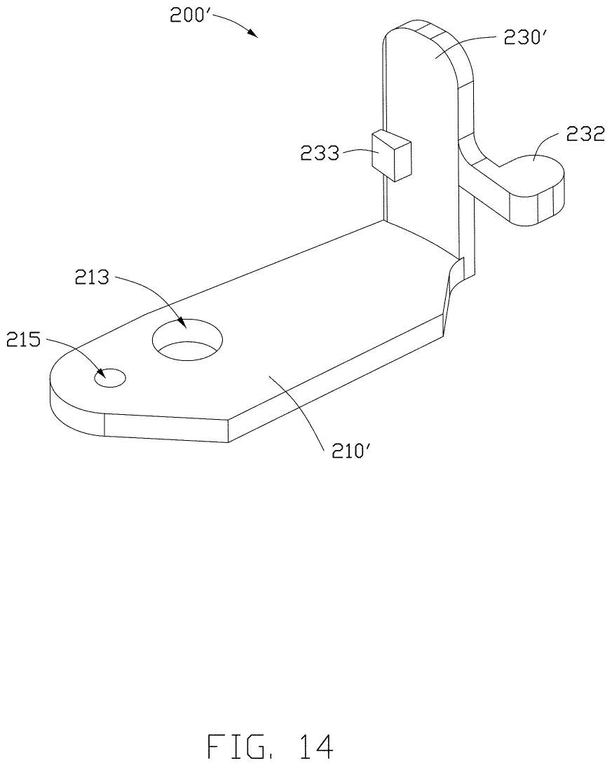

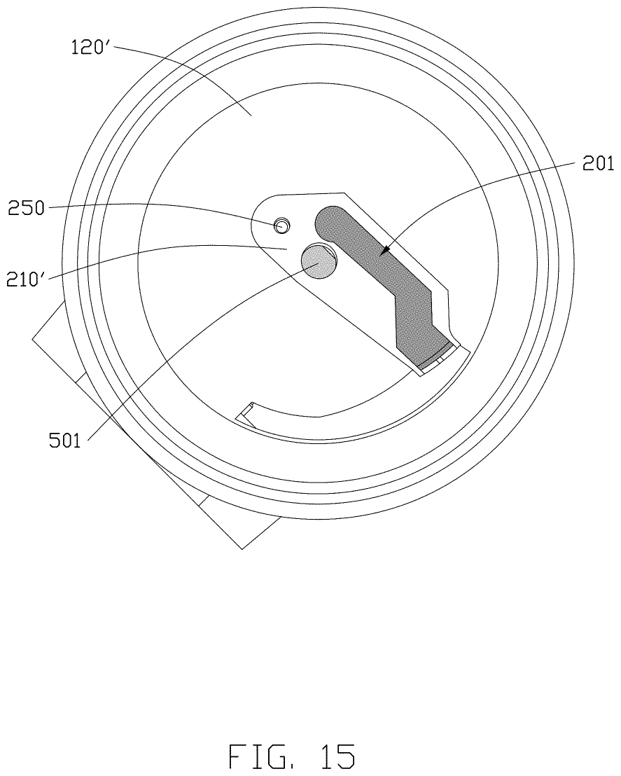

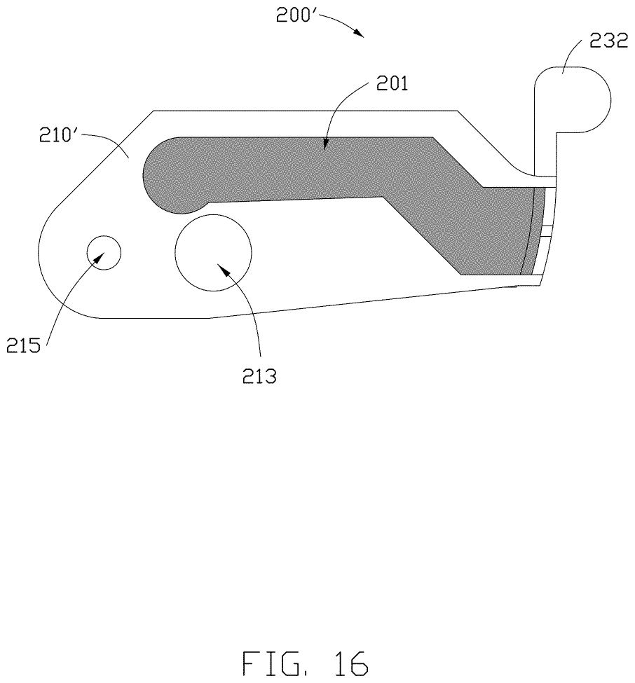

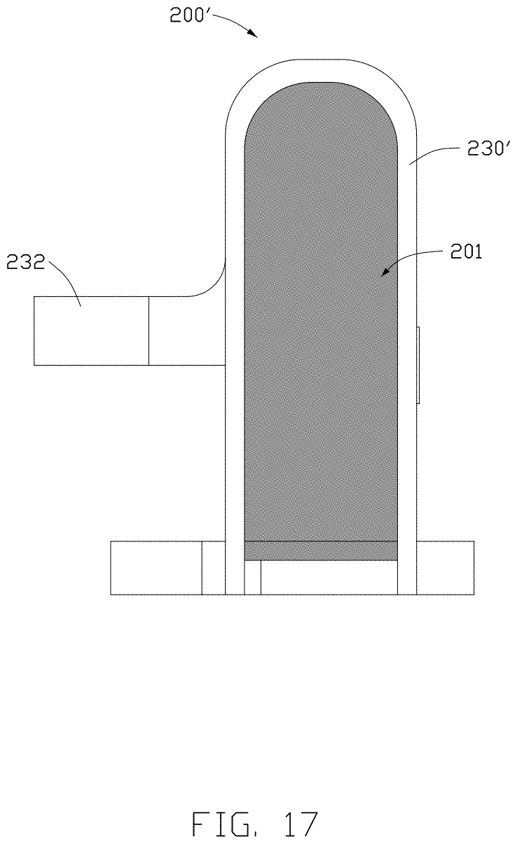

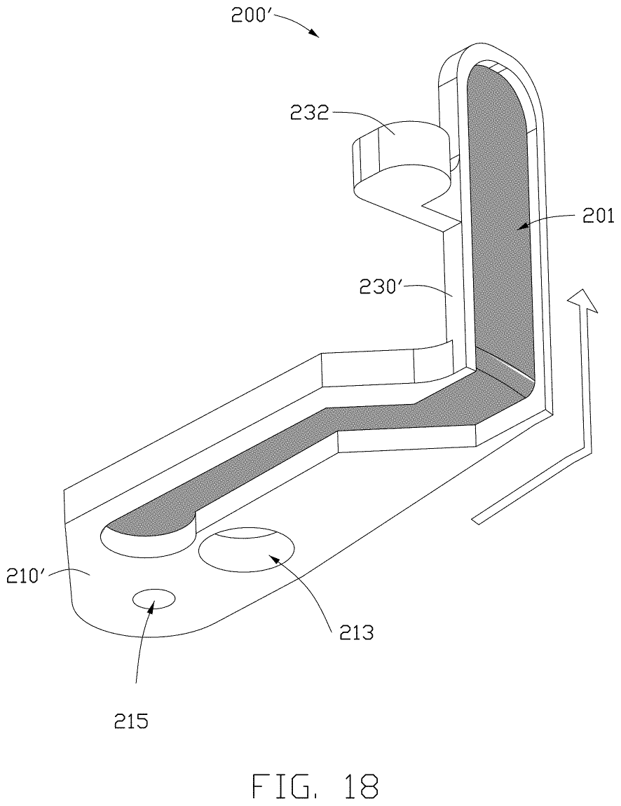

Referring to FIG. 14 to FIG. 18. In one embodiment of the present disclosure, the switching mechanism 200′ is a substantially L-shaped component. The switching mechanism 200′ includes a second base 210′ and a second extension 230′ substantially perpendicular to the second base 210′. The second extension 230′ includes a second protrusion 232 acting as the lever 150 corresponding to the sliding structure 140 of the housing 100, such that the second protrusion 232 can be placed within and slide along the sliding structure 140. Therefore, by sliding the second protrusion 232 along the sliding structure 140, a user of the stethoscope assembly 1 can move the switching mechanism 200′ between the first state and the second state to switch the stethoscope head 10′ between the digital mode (as illustrated in FIG. 12) and the conventional mode (as illustrated in FIG. 13). The second base 210′ includes a third hole 213 configured to allow the acoustic signals to travel from the through hole 301 to the microphone 501 under the digital mode. As shown in FIG. 15, the pin 250 is integrally formed at the bottom of the second deck 120′, and the second base 210′ includes the pin hole 215 configured for accommodating the pin 250, hence allowing the second base 210′ to rotate about the pin 250 between the first state and the second state. As illustrated in FIG. 16 to FIG. 18, the switching mechanism 200′ includes a substantially L-shaped acoustic channel 201 extending from the lower side of the second base 210 (as shown in FIG. 16) to the rear side of the second extension 230′ (as shown in FIG. 17). The acoustic channel 201 is configured to direct the acoustic signals from the through hole 301 to the second opening 102 of the housing 100 under the conventional mode.

Referring again to FIG. 8 and FIG. 12. When the stethoscope head 10′ is in the digital mode, the switching mechanism 200′ is at the first state, and the third hole 213 of the second base 210′ of the switching mechanism 200′ is aligned with the through hole 301 of the chestpiece 300 and the second aperture 121′ of the second deck 120′. In such configuration, the microphone 501 is exposed to the through hole 301, and therefore the microphone 501 in the second aperture 121′ receives the acoustic signals passing through the through hole 301 of the chestpiece 300 and the third hole 213 of the second base 210′. Hence, the speaker 600 in the first opening 101 plays the acoustic signals received by the microphone 501 into the tube member 20 of the stethoscope assembly 1′.

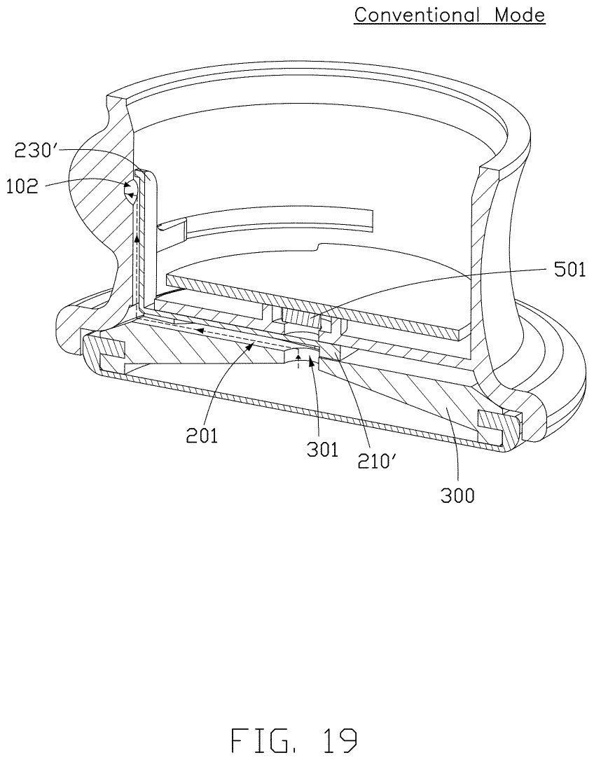

Referring to FIG. 9, FIG. 13, FIG. 19 and FIG. 20. When the stethoscope head 10′ is in the conventional mode, the switching mechanism 200′ is at the second state, and the acoustic channel 201 of the switching mechanism 200′ is aligned with the through hole 301 of the chestpiece 300 and the second opening 102 of the housing 100′. In such configuration, the microphone 501 is blocked from the through hole 301, and the acoustic channel 201 connects between the through hole 301 and the second opening 102. Therefore, as illustrated in FIGS. 19 and 20, the acoustic signals passing through the through hole 301 is directed from the lower side of the second base 210′ and the rear side of the second extension 230′ to the second opening 102 by the acoustic channel 201 without reaching the microphone 501. Hence, the acoustic signals travel to the tube member 20 from the second opening 102.

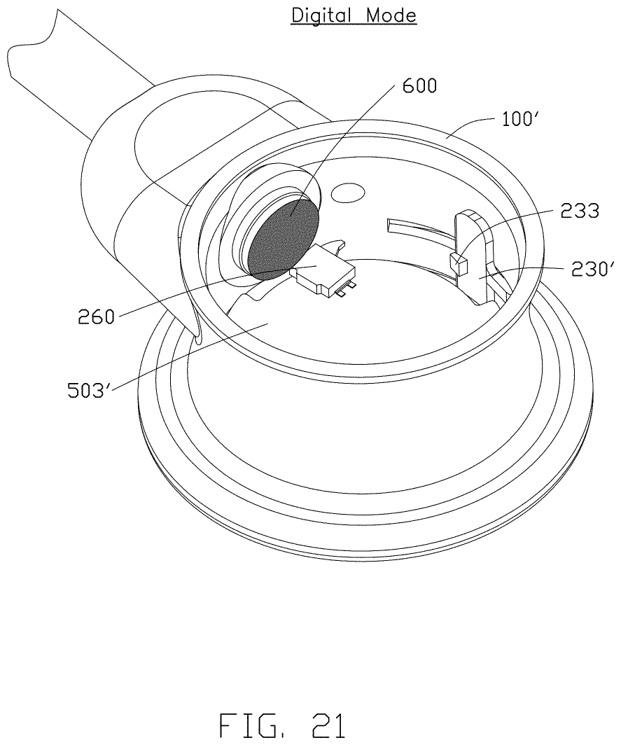

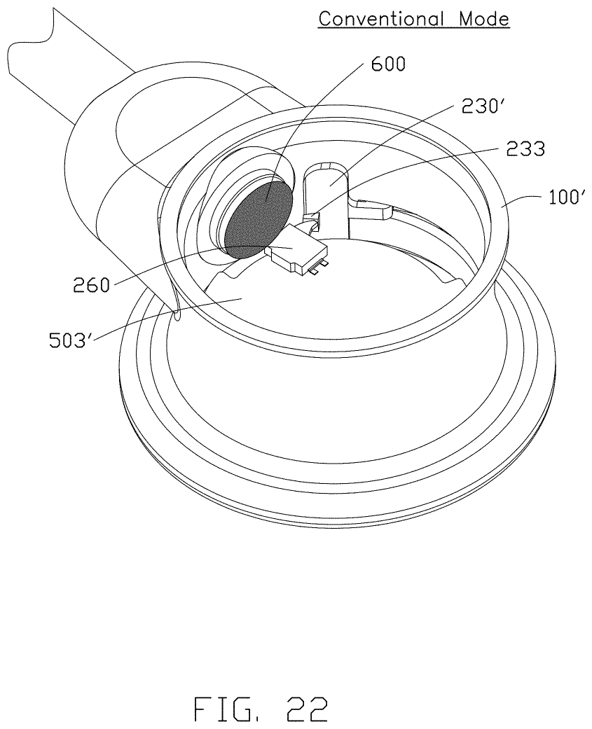

Referring to FIG. 21 and FIG. 22. In one embodiment of the present disclosure, the stethoscope head 10′ further includes a first power switch 260 electrically connected to the PCB 503 in the housing 100′. The first power switch 260 is configured to turn off at least one of the microphone 501 and the speaker 600 when engaged. Otherwise, the microphone 501 and the speaker 600 are powered by a battery electrically connected to the PCB 503′ in the housing 100′ when the first power switch 260 is not engaged. As illustrated in FIG. 22, the first power switch 260 is installed to be engaged by a third protrusion 233 on the second extension 230′ of the switching mechanism 200′ at the second state for enabling the conventional mode of the stethoscope head 10′. Alternatively, as illustrated in FIG. 21, the switching mechanism 200 at the first state is disengaged from the first power switch 260, hence the microphone 501 and the speaker 600 are powered to enable the digital mode of the stethoscope head 10′.

In another embodiment, the first power switch 260 may also be installed to be engaged by the third protrusion 233 of the extension 230 of the switching mechanism 200′ at the first state for enabling the digital mode of the stethoscope head 10′. In this case, the first power switch 260 is configured to turn on the microphone 501 and the speaker 600 when engaged. When the first power switch 260 is not engaged by the second extension 230′, the microphone 501 and the speaker 600 do not receive power from the battery, hence enabling the conventional mode of the stethoscope head 10′. Alternatively, the first power switch 260 is configured to turn on the microphone 501 or the speaker 600 when engaged. For example, the first power switch 260 may be configured to turn on the microphone 501 only, and the speaker 600 may stay powered on by the battery; vice versa. In a case where the speaker 600 stays powered on by the battery, when the first power switch 260 is not engaged by the second extension 230′, the microphone 501 does not receive power from the battery, hence enabling the conventional mode of the stethoscope head 10′; alternatively, when the first power switch 260 is engaged by the second extension 230′, the microphone 501 receives power from the battery, hence enabling the digital mode of the stethoscope head 10′.

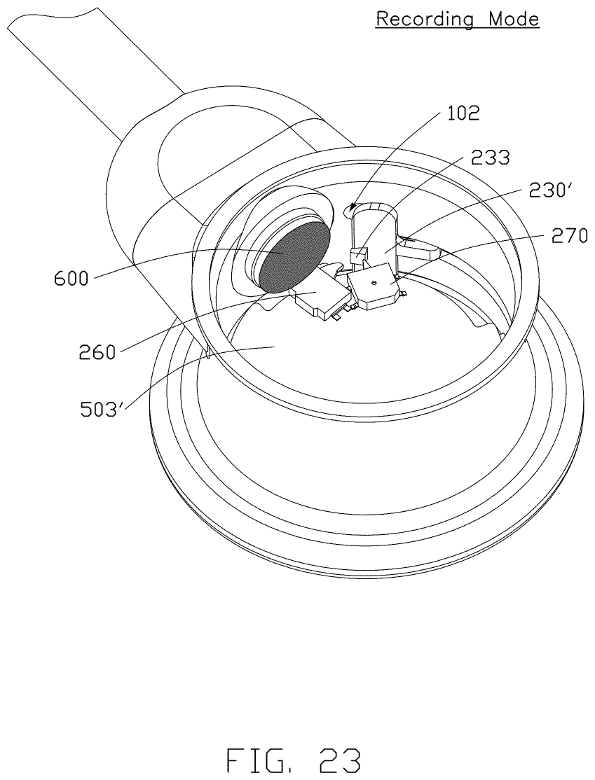

Referring to FIG. 23. In yet another embodiment of the present disclosure, the stethoscope head 10′ further includes a second power switch 270 electrically connected to the speaker 600. When engaged, the second power switch 270 is configured to turn off the speaker 600 without altering the power status of the microphone 501. In the embodiment, the second power switch 270 is installed to be engaged by the third protrusion 233 of the second extension 230′ of the switching mechanism 200′ at a third state between the first state and the second state, thus allowing a recording mode of the stethoscope head 10′. When the recording mode of the stethoscope head 10′ is enabled, both of the third hole 213 and the acoustic channel 201 are partially in fluid communication with the through hole 301; therefore a part of the acoustic signals from the through hole 301 passes the third hole 213 to be received by the microphone 501 and recorded digitally by a memory (not shown) of the PCB 503′, while the other part of the acoustic signals enters the acoustic channel 201 to be heard by a user, as in the conventional mode, via the second opening 102 and the tube member 20. Consequently, the user is able to hear the acoustic signals from the stethoscope head 10′ and record what is heard at the same time by using the recording mode.

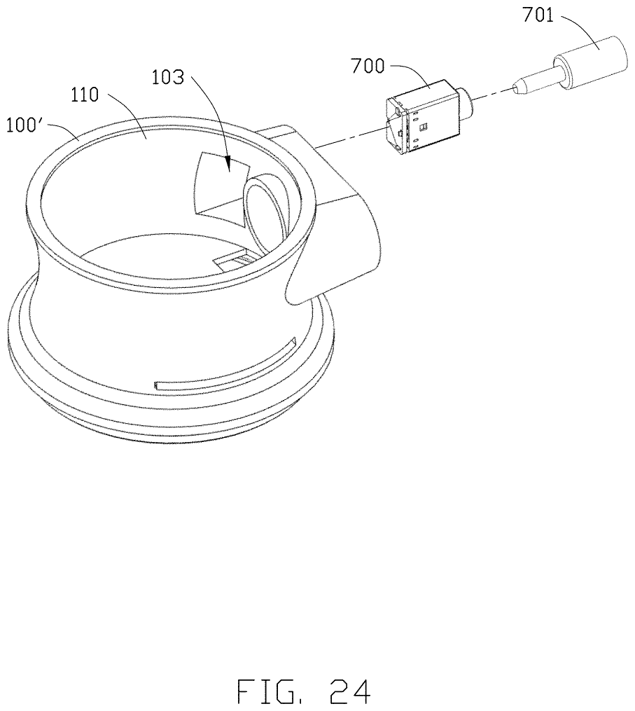

Referring to FIG. 24. In one embodiment of the present disclosure, the housing 100′ further includes a third opening 103 at the side wall 110, and the stethoscope head 10′ further includes a phone jack module 700 installed within the third opening 103. The phone jack module 700 is electrically connected to the PCB 503′, such that the acoustic signals received by the microphone 501 can be transmitted to the phone jack module 700 via the PCB 503. Therefore, the user is able to hear the acoustic signals by connecting a phone jack 701 of a headset (not shown) or an external audio device to the phone jack module 700.

It should be noted that, the lever 150, the first power switch 260, the second power switch 270, the battery, and the phone jack module 700 are independently applicable to any of the aforementioned embodiments.

In sum, the stethoscope head according to the various embodiments of the present disclosure use a power switch and a switching mechanism that can be switched between two states and is engageable to the power switch in one of the states, so as to enable the digital mode and the conventional mode. In addition, the stethoscope head can further include the recording mode by installing another power switch engageable to the switching mechanism.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A stethoscope head switchable between a digital mode and a convention al mode, comprising:
a housing;
a chestpiece, disposed below the housing and comprising a through hole at a center of the chestpiece;
a switching mechanism, disposed in the housing and above the chestpiece, and configured for switching the stethoscope head between the digital mode and the conventional mode;
a microphone, disposed in the housing and configured for receiving acoustic signals from the through hole of the chestpiece;
a speaker, disposed in the housing and configured for playing the acoustic signals received by the microphone; and
a first power switch, disposed in the housing and configured to be engaged by the switching mechanism.

2. The stethoscope head according to claim 1, wherein when the switching mechanism engages the first power switch, the first power switch turns on at least one of the microphone and the speaker, thereby allowing the stethoscope head to operate under the digital mode.

3. The stethoscope head according to claim 1, wherein when the switching mechanism engages the first power switch, the first power switch turns off at least one of the microphone and the speaker, thereby allowing the stethoscope head to operate under the conventional mode.

4. The stethoscope head according to claim 1, wherein the switching mechanism comprises a first base for accommodating the microphone and a first extension for holding the speaker.

5. The stethoscope head according to claim 4, wherein when the stethoscope head is in the digital mode, the switching mechanism is so positioned that the speaker is aligned with a first opening on the housing, the microphone is exposed to the through hole of the chestpiece, and a first aperture at a center of a first deck of the housing is not in fluid communication with the through hole, thereby enabling the microphone to receive the acoustic signals.

6. The stethoscope head according to claim 4, wherein when the stethoscope head is in the conventional mode, the switching mechanism is so positioned that the speaker is not aligned with a first opening on the housing, the microphone is blocked from the through hole of the chestpiece, and a first aperture at a center of a first deck of the housing is in fluid communication with the through hole, thereby disenabling the microphone to receive the acoustic signals.

7. The stethoscope head according to claim 1, wherein the microphone is disposed in a second aperture at a center of a second deck of the housing, and the speaker is aligned with a first opening on the housing.

8. The stethoscope head according to claim 7, wherein the switching mechanism comprises a second base having a hole, and when the stethoscope head is the digital mode, the switching mechanism is so positioned that the hole is aligned with the second aperture of the housing and the through hole of the chestpiece, thereby enabling the microphone to receive the acoustic signals.

9. The stethoscope head according to claim 7, wherein the switching mechanism comprises a second base having a hole, and when the stethoscope head is the conventional mode, the switching mechanism is so positioned that the hole is not aligned with the second aperture of the housing and the microphone is blocked from the through hole of the chestpiece, thereby disenabling the microphone to receive the acoustic signals.

10. The stethoscope head according to claim 9, wherein the switching mechanism further comprises an acoustic channel formed on a lower side of the second base of the switching mechanism, and when the stethoscope head is the conventional mode, the acoustic channel is aligned with the through hole of the chestpiece, thereby directing the acoustic signals from the through hole to a second opening on the housing.

11. The stethoscope head according to claim 10, further comprising a second power switch configured to be engaged by the switching mechanism and electrically connected to the speaker, wherein the switching mechanism engages the second power switch, the hole and the acoustic channel are partially in fluid communication with the through hole, thereby allowing the acoustic signals to be received by the microphone and be directed to a second opening on the housing simultaneously.

12. The stethoscope head according to claim 1, further comprising a guiding structure in the housing, wherein the switching mechanism extends through the guiding structure, thereby allowing the switching mechanism moving within the guiding structure to switch the stethoscope head between the digital mode and the conventional mode.

13. The stethoscope head according to claim 1, wherein the housing further comprises a sliding structure formed on a side wall, the switching mechanism comprises a lever extending through the sliding structure, thereby allowing a user to move the switching mechanism by sliding the lever along the sliding structure.

14. The stethoscope head according to claim 1, further comprising a pin, wherein the pin passes through a pin hole formed on the switching mechanism to allow the switching mechanism to rotate about the pin.

15. The stethoscope head according to claim 1, further comprising a phone jack module connected to the housing and configured for transmitting the acoustic signals received by the microphone to an external device.

16. The stethoscope head according to claim 1, wherein the switching mechanism is substantially L-shaped.
